(19) United States
(12) Patent Application Publication
(10) Pub. No.: US 2026/0053810 A1
(43) Pub. Date: Feb. 26, 2026

(54) Time-Released Caffeine Sleep Aid with Dual-Phase Formulation for Improved Sleep and Wakefulness

(71) Applicant: Caitlin Ferzli, New York, NY (US)

(72) Inventor: Caitlin Ferzli, New York, NY (US)

(21) Appl. No.: 18/813,030

(22) Filed: Aug. 23, 2024

(51) Int. Cl. A61K 31/522; A61K 31/197; A61K 31/4045; A61K 31/405; A61K 31/714; A61K 33/06; A61K 36/84

(52) U.S. Cl. CPC A61K 31/522; A61K 31/197; A61K 31/4045; A61K 31/405; A61K 31/714; A61K 33/06; A61K 36/84

(57) ABSTRACT

A dual-phase sleep aid supplement designed to promote restful sleep and enhance wakefulness. The supplement includes an immediate release layer containing sleep-promoting ingredients and a delayed release layer of caffeine designed to release after 6-8 hours. The formulations include options with melatonin or vitamin B12 and tryptophan.

### BACKGROUND

This invention relates to dietary supplements, specifically to sleep aids designed to promote restful sleep and enhanced wakefulness through a dual-phase formulation that includes time-released caffeine.

Traditional sleep aids focus solely on promoting sleep without addressing the need for wakefulness in the morning. There is a need for a sleep aid that not only helps users fall asleep and stay asleep but also provides a timed release of caffeine to enhance morning alertness.

### SUMMARY OF INVENTION

The present invention provides a dual-phase sleep aid supplement designed to release sleep-promoting ingredients immediately and caffeine after a delay of 6-8 hours. This approach ensures restful sleep and wakefulness in the morning.

### DETAILED DESCRIPTION

### Materials and Methods

Time-Released Caffeine: Caffeine is encapsulated in materials that dissolve slowly, such as ethylcellulose or other biocompatible polymers, to ensure a delayed release.
Immediate Release Ingredients: Two formulations are proposed:

### Melatonin Formulation

Melatonin: To regulate sleep-wake cycles.
Valerian Root: To promote relaxation.
Magnesium: To aid muscle relaxation.
L-Theanine: To reduce stress and promote calmness.

### B12 and Tryptophan Formulation

Vitamin B12: To support circadian rhythms.
Tryptophan: A precursor to serotonin and melatonin.
Magnesium: To aid muscle relaxation.
L-Theanine: To reduce stress and promote calmness.

### Mechanism

Delayed Release Mechanism: Microencapsulation, multi-layer tablets, or matrix systems are used to control the release of caffeine.

### Formulation

### Melatonin Formulation

Immediate Release Layer: Melatonin (1 mg), Valerian Root Extract (200 mg), Magnesium (100 mg), L-Theanine (50 mg).
Delayed Release Layer: Microencapsulated Caffeine (100 mg) designed to release after 6-8 hours.

### B12 and Tryptophan Formulation

Immediate Release Layer: Vitamin B12 (500 mcg), Tryptophan (500 mg), Magnesium (100 mg), L-Theanine (50 mg).
Delayed Release Layer: Microencapsulated Caffeine (100 mg) designed to release after 6-8 hours.
Benefits: The dual-phase formulation ensures that users receive sleep support initially and a timed release of caffeine to help wake up refreshed and alert.

Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A sleep aid supplement comprising microencapsulated caffeine, wherein the encapsulating material is a polymer selected from the group consisting of ethylcellulose and other biocompatible polymers, designed to degrade after a delay of 6-8 hours.

2. The supplement of claim 1, further comprising an immediate release layer containing melatonin, valerian root extract, magnesium, and L-theanine.

3. The supplement of claim 1, further comprising an immediate release layer containing vitamin B12, tryptophan, magnesium, and L-theanine.

4. The supplement of claim 1, in the form of a capsule.

5. The supplement of claim 1, in the form of a tablet.

6. The supplement of claim 1, in the form of a gummy.
